(19) United States
(12) Patent Application Publication
(10) Pub. No.: US 2025/0022293 A1
(43) Pub. Date: Jan. 16, 2025

(54) MANAGEMENT SYSTEM FOR GENERATING AND DISTRIBUTING CELL IMAGE-BASED NFT

(71) Applicant: INDUSTRY-ACADEMIC COOPERATION FOUNDATION, YONSEI UNIVERSITY, Seoul (KR)

(72) Inventor: Jinkee HONG, Seoul (KR)

(73) Assignee: INDUSTRY-ACADEMIC COOPERATION FOUNDATION, YONSEI UNIVERSITY, Seoul (KR)

(21) Appl. No.: 18/714,632

(22) PCT Filed: Nov. 8, 2022
(86) PCT No.: PCT/KR2022/017487

§ 371 Date: Oct. 3, 2024

(30) Foreign Application Priority Data
Nov. 9, 2021 (KR) 10-2021-0153140
Nov. 7, 2022 (KR) 10-2022-0147357

(51) Int. Cl. G06V 20/69; G06F 21/10; H04L 9/32

(52) U.S. Cl. CPC G06V 20/693; G06F 21/108; H04L 9/3213

(57) ABSTRACT

The present disclosure relates to a management system for generating and distributing a cell image-based NFT including a cell image generation unit that generates an image from a collected cell, an NFT issuance unit that converts the cell image into an NFT image and stores and manages the NFT image, and an NFT transaction unit that brokers an online transaction for the issued NFT, and a service providing method thereof, and may assign an asset value to a vital image to meet the demand for owning the image.

### TECHNICAL FIELD

The present disclosure provides a management system for generating and distributing a cell image-based NFT and a service providing method thereof.

### BACKGROUND ART

As a blockchain technology market expands around the world, a non fungible token (NFT)-related technology is rapidly growing to implement proof of ownership not only for digital art or digital fashion items for virtual world avatars, but also for various goods in the real world.

The NFT is a type of digital registration certificate stored on the blockchain. The NFT includes the Internet address where the original is stored, owner identity information, and smart contract information, and is used as a means to buy and sell ownership of digital assets. The blockchain technology may be used to track a person who owns the NFT, and satisfy those who have a desire to own digital assets because counterfeiting and duplication of digital assets is impossible.

Until now, the NFT have been used as a means to satisfy people's desire to show off by purchasing the NFT for investment purposes in collectibles such as artwork and stamps or by registering the NFT as a profile on SNS. Alternatively, the NFT has been used as an admission ticket to participate in a project, community, or performance, or used for purposes such as applying the NFT to an authenticity authentication system in connection with physical products.

In addition, the NFT technology is currently being used usefully in the trading of game items or digital art, but the usefulness is not permanently guaranteed because the game items become useless when the related game services end. In addition, since the digital art can be completely duplicated, the ownership of the original file of the digital art has limited utility other than personal satisfaction, and since there are many cases where there is a separate copyright holder, the owner of digital art may have significant restrictions on its use in such cases.

In this way, the digital assets that the NFT may demonstrate their potential are still unclear and impermanent. Therefore, the development of various services is necessary so that the NFT may have greater influence with guaranteed functions or values.

### DISCLOSURE

### Technical Problem

An object of the present disclosure is to own vital images such as humans or animals, produce and sell images created by collecting cells over time, like a biography, or secure diversification of business models of the digital assets, such as combining the vital images with other products, by converting cell images derived from living organisms into digital assets.

### Technical Solution

In one general aspect, a management system for generating and distributing a cell image-based NFT includes: a cell image generation unit 100 that generates an image from a collected cell; an NFT issuance unit 200 that converts the cell image into an NFT image and stores and manages the NFT image; and an NFT transaction unit 300 that brokers an online transaction for the issued NFT.

The NFT image may be a static image or dynamic data to which sound is added.

The cell image generation unit may acquire the collected cell as an imaging result using a fluorescence microscopy, a confocal microscopy, a phase-contrast microscopy, an electron microscopy, bright field imaging, or a combination thereof.

The NFT issuance unit may include an NFT conversion unit 210 that generates an NFT image, an encryption key, and a decryption key, an NFT storage management unit 220 that stores the generated NFT image, encryption key, and decryption key, and an NFT providing unit 230 that transfers the NFT image, the encryption key, and the decryption key when the transaction of the NFT image is completed.

The NFT transaction unit may include an application unit 310 that provides the NFT image and basic information of a cell image provider and applies for online transaction of the NFT image, and a processing unit 320 that processes NFT payment according to the NFT transaction application details and provides the NFT image, the encryption key and the decryption key to an NFT buyer when the payment is completed.

The NFT image may further include a digital image other than the cell image.

In another general aspect, a service providing method for generating and distributing a cell image-based NFT includes: acquiring a cell image from a collected cell; issuing a blockchain-based NFT indicating ownership of the cell image so that the cell image has a unique token type used in a blockchain-based network; receiving a request related to the cell image from a service user terminal connected to the blockchain-based network; and providing the NFT to the service use terminal in response to the received request.

The cell image may be derived from a cell collected from an oral cavity or urine.

### Advantageous Effects

According to the present disclosure, it is possible to produce vital images such as humans or animals into NFT, own and sell the NFT, produce and sell images created by collecting cells over time, like a biography, or provide various business models such as combining the vital images with other products.

### BRIEF DESCRIPTION OF THE DRAWINGS

FIG. 1 is a diagram illustrating the overall configuration of a management system for generating and distributing a cell image-based NFT according to an embodiment of the present disclosure.

FIG. 2 is a diagram illustrating an NFT image and use according to an embodiment of the present disclosure.

FIG. 3 is a block diagram illustrating a configuration of an NFT issuance unit according to an embodiment of the present disclosure.

FIG. 4 is a block diagram illustrating a configuration of an NFT transaction unit according to an embodiment of the present disclosure.

FIG. 5 is a flowchart illustrating the entire process of a service providing method for generating and distributing a cell image-based NFT according to an embodiment of the present disclosure.

### BEST MODE

Hereinafter, a management system for generating and distributing a cell image-based NFT and a service providing method thereof according to the present disclosure will be described in detail with reference to the attached drawings so that a person skilled in the art can easily perform it.

Technical terms and scientific terms used in the present specification have the general meaning understood by those skilled in the art to which the present disclosure pertains unless otherwise defined, and a description for the known function and configuration unnecessarily obscuring the gist of the present disclosure will be omitted in the following description and the accompanying drawings.

The present disclosure may be implemented in various different forms and is not limited to embodiments or drawings described herein. In the drawings, portions unrelated to the description will be omitted to clearly describe the present disclosure, and similar portions will be denoted by similar reference numerals throughout the specification. The drawings are provided as examples to enable the idea of the present disclosure to be sufficiently conveyed to those skilled in the art to which the present disclosure pertains, the present disclosure is not limited to the presented drawings and may be embodied in other forms, and the drawings may be exaggerated to clarify the spirit of the present disclosure.

The singular forms used in the specification are intended to include the plural forms as well, unless the context specifically dictates otherwise.

In addition, “Including” mentioned herein is an open-ended description having an equivalent meaning to expressions such as “comprising,” “containing,” “having,” “characterizing,” and elements, materials, or processes not listed additionally are not excluded.

In addition, terms “ . . . unit,” “module,”, and the like, described in the specification refer to a processing unit of at least one function or operation and may be implemented by hardware or software or a combination of hardware and software.

The management system for generating and distributing a cell image-based NFT and a service provision method thereof according to an embodiment of the present disclosure may convert cell images of various living organisms such as humans, animals, and plants into non-fungible unique digital data and then trade and distribute the digital data, and thus, may be used to sell and utilize the cell images.

The present disclosure relates to a management system for generating and distributing a cell image-based NFT, including: a cell image generation unit that generates an image from a collected cell; an NFT issuance unit that converts the cell image into an NFT image and stores, manages, and provides the NFT image; and an NFT transaction unit that brokers an online transaction for the issued NFT.

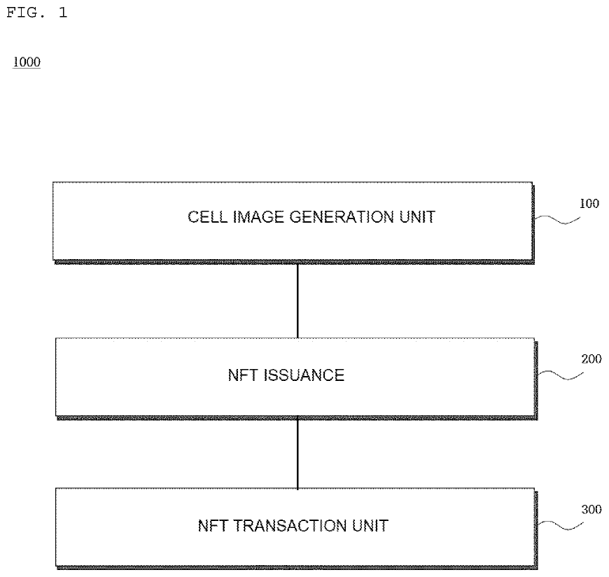

FIG. 1 is a diagram illustrating the overall configuration of a management system 1000 for generating and distributing a cell image-based NFT according to an embodiment of the present disclosure.

Referring to FIG. 1, the management system 1000 for generating and distributing a cell image-based NFT according to an embodiment of the present disclosure generates cells obtained from cell donors as images and provides the generated images to the NFT issuance unit. The cell images may be converted into the NFT images through an automated image generation algorithm, encrypted, and then combined with other encrypted information and stored on a blockchain network. The issued NFT may be distributed and traded using cryptocurrency or virtual currency through online markets. The NFT transaction may be made through the sale of ownership or usage rights, and even the encrypted information included in the NFT image may be transferred during the transaction.

The NFT creator may earn more profits as the NFT is more traded or utilized on the blockchain network, which may be in the form of receiving an air drop through cryptocurrency or virtual currency.

The cell image-based NFT according to the present disclosure may be traded through all consumers who need to purchase NFTs, but the distribution and transaction of the NFTs may be limited to consumers whose identities have been verified through a blockchain identity authentication system.

Looking at the specific configuration of the management system 1000 for generating and distributing a cell image-based NFT, the cell image generation unit 100 may acquire images of living cells through various cell imaging methods, including a phase-contrast microscopy, fluorescence imaging of single molecule or organelles of a cell, a confocal microscopy, live cell imaging, an electron microscopy, etc., for the collected cells.

The collected cells may be derived from secretions such as skin, oral tissue, fat tissue, blood, or urine, but are specifically obtained through non-medical procedures, and may be cells collected from, for example, skin, oral tissue, or urine. More specifically, it may be preferable that cells be derived from urine because they do not require medical procedures during the collection process and do not cause pain to donors.

For example, the urine-derived cells may be collected, stem cells may be obtained from the collected urine-derived cells, the stem cells may be differentiated, and then the images of the differentiated cells may be obtained.

The cell images may be obtained from all cells such as cardiac muscle tissue, nerve cell bundles, and vascular cell tubes, and may include all proliferation, differentiated states, etc.

The cell images may be not only static images, but also dynamic data that may confirm cell motions, cell movement, etc. In addition, the cell image may be a static image or dynamic data with sound added. As a specific example, when the cell is a cardiac muscle cell, the cell image may be dynamic data in which movement such as a heartbeat is recognized, and/or may be a static image or dynamic data to which a heartbeat sound is added.

The cell image may be used alone, and may be created in a fused form with other digital images, sounds, and other digital effects other than cells. The cell image may include meta data, which may include descriptions of the images, sounds, and other digital effects, production processes, and vital information. This information may be stored in any suitable form.

In the present disclosure, the NFT image may be a combination of a cell image and other works. Specifically, the digital content such as paintings or music may be converted into an NFT image by combining with the cell image. As a non-limiting example of the present disclosure, the NFT image may be a combination of cell images and other digital images. In addition, other analysis information on life (genome information, metabolic information, etc.) may be added and stored in the form of meta data.

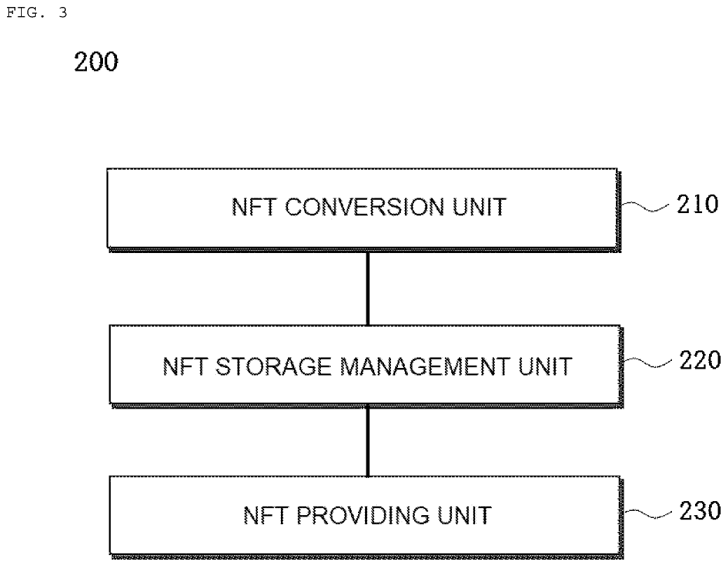

Referring to FIG. 3, the NFT issuance unit 200 may include an NFT conversion unit 210 that converts a cell image into an NFT image and generates an encryption key and a decryption key during the conversion process of the NFT image, an NFT storage management unit 220 that stores the NFT image, the encryption key, and the decryption key generated through the NFT conversion unit on a blockchain network, and an NFT providing unit 230 that transfers the NFT image, the encryption key, and the decryption key stored on the blockchain network through the NFT transaction unit when the transaction of the NFT image is completed.

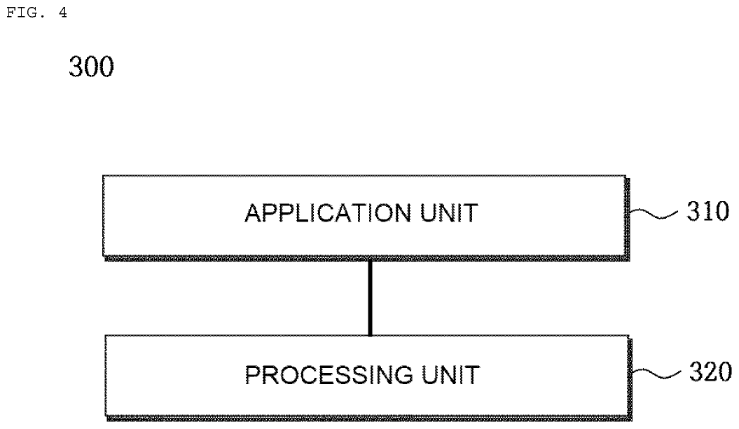

Referring to FIG. 4, the NFT transaction unit 300 may include an application unit 310 that provides the NFT image and basic information of a cell image provider and applies for online transaction of at least one of usage right and ownership of the NFT image, and a processing unit 320 that processes NFT payment using cryptocurrency according to the NFT transaction application details and provides the NFT image, the encryption key and the decryption key applied through the application unit to an NFT buyer when the payment is completed.

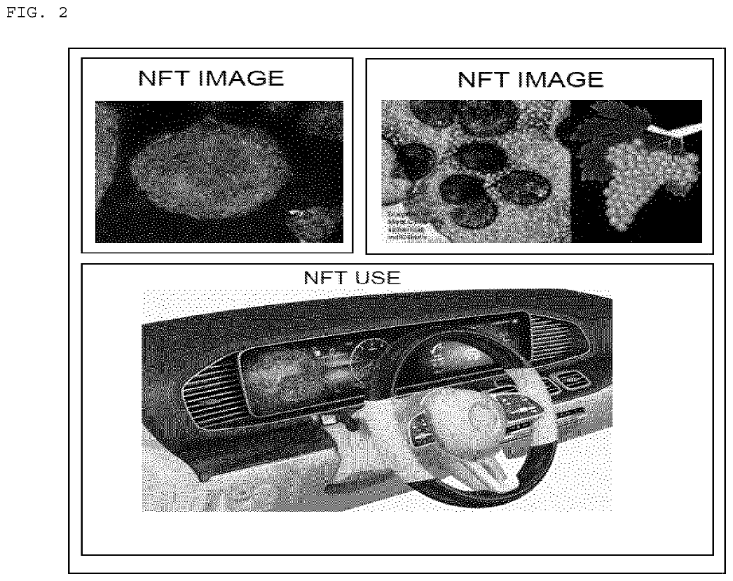

According to the present disclosure, the NFT may be used in combination with a product including a display unit. As a specific example, reference may be made to the use of NFT illustrated in FIG. 2. By registering an NFT converted from an image of a horse's heart muscle cell on a car instrument panel display unit, an image of a NFTied horse's heart muscle cell may be displayed on the display at the same time as a car is started. Therefore, when purchasing a product, customizing services may be provided to an NFT image licensee or owner. This can be the creation of a personalized product that contains my vital information, which is only one in the world, and if various technologies develop, it may also be used for digital identification purposes.

In addition, the cell image-based NFT according to the present disclosure may be used in various ways on metaverse platforms, and the scope of use may be determined depending on users. For example, it may be used as a profile picture on SNS, as a game item, as applying to an avatar, and as authentication or authorization.

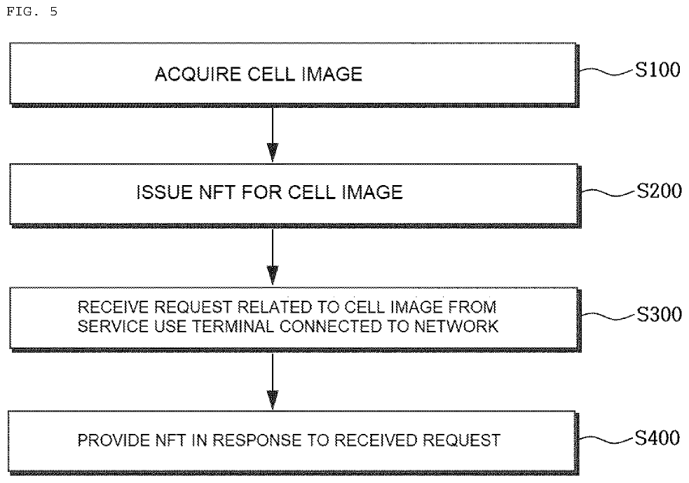

FIG. 5 is a flowchart illustrating the entire process of service providing method for generating and distributing a cell image-based NFT according to another embodiment of the present disclosure.

Referring to FIG. 5, a service providing method for generating and distributing a cell image-based NFT includes: (S100) acquiring a cell image from a collected cell; (S200) issuing a blockchain-based NFT indicating ownership of the cell image so that the cell image has a unique token type used in a blockchain-based network; (S300) receiving a request related to the cell image from a service user terminal connected to the blockchain-based network; and (S400) providing the NFT to the service use terminal in response to the received request.

In an example of the service providing method for generating and distributing a cell image-based NFT of the present disclosure, the cell images may be images acquired from cells derived from endangered plants and animals. By converting the image into the NFT and providing it, it may contribute to spreading awareness to preserve life at the risk of extinction from threats such as environmental pollution, and may be used to improve the profits of public projects.

In relation to embodiments of the present disclosure, the cryptocurrency used for the NFT conversion and transaction may be Ethereum, Polygon, self-issued coins, tokens, or the like, but is not necessarily limited thereto.

What has been described above is only an example for carrying out the management system for generating and distributing a cell image-based NFT and service providing method thereof according to the present disclosure, and is not limited thereto. As claimed in the claims below, it is said that the technical idea of the present disclosure is to the extent that various modifications can be made by anyone skilled in the art without departing from the gist of the present disclosure.

### DESCRIPTION OF REFERENCE SIGNS

1000: Management system for generating and distributing a cell image-based NFT
100: Cell image generation unit
200: NFT issuance unit
210: NFT conversion unit
220: NFT storage management unit
230: NFT providing unit
300: NFT transaction unit
310: Application unit
320: Processing unit

1. A management system for generating and distributing a cell image-based NFT, comprising:
a cell image generation unit that generates an image from a collected cell;
an NFT issuance unit that converts the cell image into an NFT image and stores and manages the NFT image; and
an NFT transaction unit that brokers an online transaction for the issued NFT.

2. The management system of claim 1, wherein the NFT image is a static image or dynamic data to which sound is added.

3. The management system of claim 1, wherein the cell image generation unit acquires the collected cell as an imaging result using a fluorescence microscopy, a confocal microscopy, a phase-contrast microscopy, an electron microscopy, bright field imaging, or a combination thereof.

4. The management system of claim 1, wherein the NFT issuance unit includes an NFT conversion unit that generates an NFT image, an encryption key, and a decryption key, an NFT storage management unit that stores the generated NFT image, encryption key, and decryption key, and an NFT providing unit that transfers the NFT image, the encryption key, and the decryption key when the transaction of the NFT image is completed.

5. The management system of claim 1, wherein the NFT transaction unit includes an application unit that provides the NFT image and basic information of a cell image provider and applies for online transaction of the NFT image, and a processing unit that processes NFT payment according to the NFT transaction application details and provides the NFT image, the encryption key and the decryption key to an NFT buyer when the payment is completed.

6. The management system of claim 1, wherein the NFT image further includes a digital image other than the cell image.

7. A service providing method for generating and distributing a cell image-based NFT, comprising:
acquiring a cell image from a collected cell;
issuing a blockchain-based NFT indicating ownership of the cell image so that the cell image has a unique token type used in a blockchain-based network;
receiving a request related to the cell image from a service user terminal connected to the blockchain-based network; and
providing the NFT to the service use terminal in response to the received request.

8. The service providing method of claim 7, wherein the cell image is derived from a cell collected from an oral cavity or urine.

9. The service providing method of claim 7, wherein the NFT image is a static image or dynamic data to which sound is added.

10. The service providing method of claim 7, wherein the cell image acquires the collected cell as an imaging result using a fluorescence microscopy, a confocal microscopy, a phase-contrast microscopy, an electron microscopy, bright field imaging, or a combination thereof.
